(19) United States
(12) Plant Patent Application Publication
(10) Pub. No.: US 2006/0101551 P1
(43) Pub. Date: May 11, 2006

(54) Plant variety of Magnolia figo named 'Purple Queen'

(50) MAGNOLIA FIGO; MAGNOLIA

(76) Inventor: Akira Shibamichi, Kawaguchi City (JP)

Correspondence Address: CHRISTIE, PARKER & HALE, LLP, PO BOX 7068, PASADENA, CA, 91109-7068, US

(21) Appl. No.: 10/984,433

(22) Filed: Nov. 8, 2004

(51) Int. Cl. A01H 5/00

(52) U.S. Cl. PLT/223

(57) ABSTRACT

A new plant variety of Magnolia figo named ‘Purple Queen’ distinguished from its parents and all other varieties of Magnolia figo by its dark red colored flowers and darker colored foliage.

### BOTANICAL CLASSIFICATION

Magnolia figo (Lour.) DC

### VARIETAL NAME

‘Purple Queen’

### BACKGROUND OF THE INVENTION

The present invention relates to a new and distinct variety of Magnolia figo (Lour.) DC which was developed in a controlled breeding program in Kawaguchi City, Japan by the originator Mr. Akira Shibamichi. The varietal denomination of the new variety is ‘Purple Queen’.

The classification for Magnoliaceae has recently undergone changes (based on recent reclassification by Figlar & Nooteboom, 2004). Magnolia figo, was previously named Michelia figo. The family Magnoliaceae comprises about 200 species. Magnolia figo is included in the subsection Michelia which contains approximately 64 species.

Magnolia figo is native to southeast China. It generally is an evergreen shrub or small tree to 4.5 meters tall. Leaf color is dark green depending on light intensity and cultural conditions. Leaves are densely branched, 10 cms long and approximately 5 cms wide; elliptic to oblong. Flower color is ivory to light yellow, with margins occasionally rose to purple. Flowers mostly on proleptic brachyblasts, rarely terminal on long shoots. The gynoecium is stipitate. Latrorse anther dehiscence. Fruits cylindrical, apocarpus or occasionally ellipsoidal syncarpus. Usually 2 to 6 (to many) ovules per carpel.

### SUMMARY OF THE INVENTION

The new variety was discovered as an open pollinated seedling in a controlled planting of Magnolia figo. The new variety differs from its parents in having dark red colored flowers and darker colored foliage. Asexual reproduction of the new variety by cuttings and grafting has confirmed that the distinctive characteristics of the new variety are stable and reproduced true to type in successive generations of asexual propagation.

### COMPARISON WITH PARENTS

Magnolia figo ‘Purple Queen’ is distinguished from its parents and all other varieties of Magnolia figo of which I am aware by its dark red colored flower and darker colored foliage.

### BRIEF DESCRIPTION OF ILLUSTRATIONS

The accompanying illustrations show a specimen of the new cultivar in a photo illustration as true to color as is reasonably possible to make in an illustration of this character.

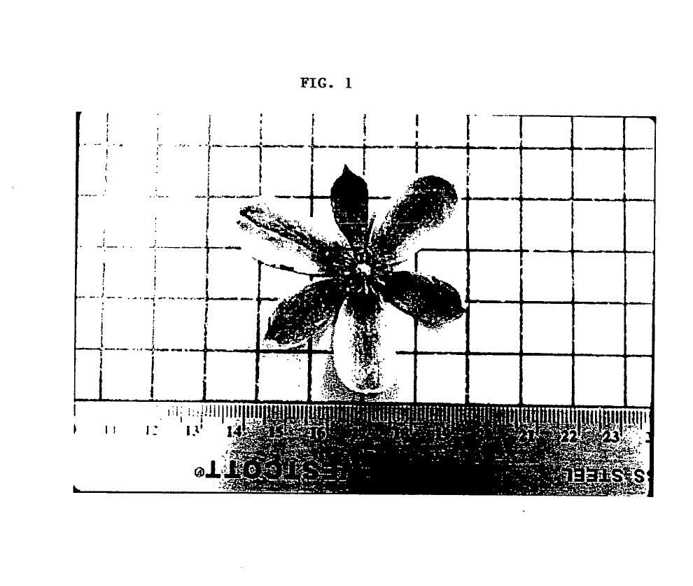

FIG. 1 illustrates the plants flower

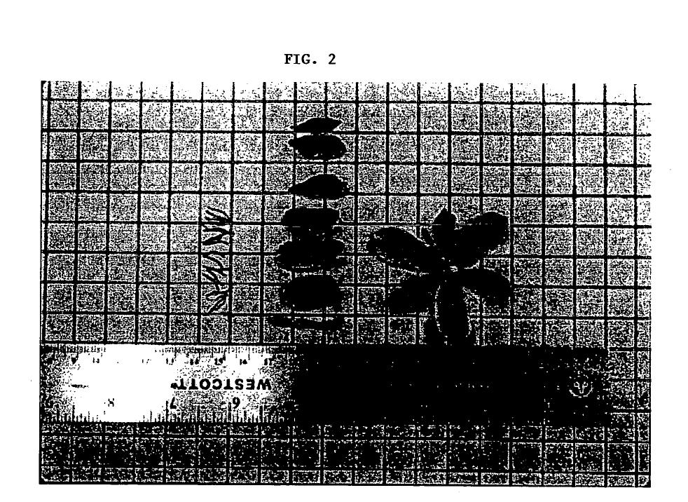

FIG. 2 illustrates the plants flowers dissected

### DETAILED DESCRIPTION OF THE NEW VARIETY

Magnolia figo ‘Purple Queen’ has not been observed under all possible environmental, cultural and light conditions. The following observations and descriptions are of plants grown in Vacaville, Calif. In this description, color references are to The Royal Horticultural Society Colour Chart (2001) and terminology used in the color descriptions herein refers to plate numbers in this color chart. Phenotypic expression may vary with light intensity, cultural and environmental conditions.
Classification:
   Botanical.—Magnolia figo.
   Parentage.—Mother Plant: Magnolia figo (unnamed, unpatented) Father Plant: Magnolia figo (unnamed, unpatented).
   Propagation.—Vegetative cuttings and grafting.
Plant:
   Size.—2.4 to 4.5 m tall and wide.
   Habit.—Upright, pyramidal compact evergreen shrub.
Branches:
   Branching.—Densely branched.
   Lateral branches.—Typical No. Branches: 55 to 80 Observed No. Branches: 40 to 60 Size: 60 to 100 cm long Diameter: 0.5 to 1.5 cm at trunk Color: Brown Group N200A-B Color of New Shoots: Near Brown Group 200A-C.
Leaf:
   Shape.—Oblong-elliptic, rarely ovate-elliptic, Apex: Acuminate or short acuminate, Base: obtuse.
   Leaf size.—10.5-19.5 cm long 4-5.5 cm wide.
   Arrangement.—Alternate.
   Margin.—Entire.
   Texture/substance.—Adaxial: Glabrous Abaxial: Slightly pubescent.
   Color.—The following Color Readings are of mature specimens grown outside in Vacaville, Calif. Young Foliage: Upper side: Near Green Group 141A-B Under side: Near Yellow-Green Group 147B Mature Foliage Upper side: Near Green Group 139A Under side: Near Yellow-Green Group 146A.
   Venation.—Pattern: Upper Surface: Pinnate Under Surface: Pinnate Coloration New Foliage: Upper Surface: Near Green Group 141A-B Under Surface: Near Yellow-Green Group 147B Mature Foliage: Upper Surface: Near Green Group 139A Under Surface: Near Yellow-Green Group 146A.
   Petioles.—Size: 7-10 mm Coloration: Near Yellow-Green Group 147B Texture: Pubescent.
   Internodes.—30-40 cm.
   Hardiness.—USDA Zone 7 (0° F. to 10° F.).
   Vigor.—Moderate.
   Pests/diseases.—None noted.
Infloresence:
   Bloom period.—Outdoors: March-April Greenhouse: February-March.
   Flower form.—Single.
   Flower arrangement.—Solitary and axillary.
   Flower size.—5.5-6 cm in diameter.
   Shape of inflorescence.—Solitary flowers only.
   Fragrance.—Slight banana scent.
   Lastingness of.—Individual Blooms: 2-3 days.
   Tepals.—Number: 6 Shape: Obovate Size: 3-3.5 cm long, 1-1.5 cm wide. Margin: Entire Curvature: Slightly cupped Arrangement: Whorled Color: Purple Group N79 A-D, fading to Purple Group N77 B Abaxial midrib: Yellow-Green Group 145 AB Margin: White Group 155 B.
Reproductive organs:
   Androecium.—Stamens: 25-30 Size: 10 mm Anther Shape: Introrse, longitudinally dehiscent Color: Near White Group 155 A-C Size: 6 mm Pollen Amount: Numerous Pollen Color: Near White Group 155A-C Filament Size: 4 mm Color: Greyed-Purple Group N186A.
   Gynoecium.—Pistil Length: 5 mm Stigma Color: Near Purple Group N77A Style Color: Near Purple Group N77B fading to Yellow-Green Group 145C Ovary Color: Near Yellow-Green Group 145A-B.
   Fruit.—Type: Aggregate, spicate Typical amount: Approximately 30 Observed amount: None observed.

I claim:

1. A new plant variety of Magnolia figo of the variety substantially as shown and described.
